(19) United States
(12) Patent Application Publication
(10) Pub. No.: US 2026/0115156 A1
(43) Pub. Date: Apr. 30, 2026

(54) INJECTABLE COMPOSITION INCLUDING HYDROGEL CONTAINING ANESTHETIC AGENT

(71) Applicants: Korea University Research and Business Foundation, Seoul (KR); The General Hospital Corporation, Boston, MA (US)

(72) Inventors: Hyun Koo Kim, Seoul (KR); Chang Geun Kim, Seoul (KR); Ok Hwa Jeon, Seoul (KR); Byeong Hyeon Choi, Seoul (KR); Kyungsu Kim, Seoul (KR); Eunbi Jun, Seoul (KR); Ga-Yoon Kim, Seoul (KR); Hak Soo Choi, Needham, MA (US); Seung Hun Park, Boston, MA (US)

(21) Appl. No.: 19/372,878

(22) Filed: Oct. 29, 2025

Related U.S. Application Data
(60) Provisional application No. 63/713,677, filed on Oct. 30, 2024.

(51) Int. Cl. A61K 31/167; A61K 9/00; A61K 31/196; A61K 47/61; A61K 47/69; A61P 23/02

(52) U.S. Cl. CPC A61K 31/167; A61K 9/0019; A61K 31/196; A61K 47/61; A61K 47/6903; A61P 23/02

(57) ABSTRACT

Provided is an injectable composition including a compound configured to form nanoparticles, an anesthetic agent, and a hyaluronic acid hydrogel. According to the present disclosure, the injectable composition is injected in a liquid formulation and gelated in the body to prevent rapid decomposition, sustained-release the anesthetic agent for a long period of time, and locally remain in the body for a long period of time. In addition, the injectable composition may provide a long-term, sustained pain relief effect through the stable sustained-release of lidocaine.

The present application claims priority to U.S. Provisional Application No. 63/713,677, filed Oct. 30, 2024, which is incorporated herein by reference in its entirety.

### TECHNICAL FIELD

The present disclosure relates to an injectable composition including a hydrogel containing an anesthetic agent, a method of preparing the same, and the like.

### BACKGROUND ART

Lidocaine is a drug widely used as a local anesthetic and analgesic agent, and has been developed into various forms of preparations for pain relief. However, due to the short half-life and rapid metabolism in vivo of lidocaine, there are limitations in situations where continuous drug delivery is required. To solve these problems, a drug delivery system capable of slowly releasing lidocaine is required, and in particular, a drug delivery medium based on a hydrogel has attracted attention.

The hydrogel is a polymer network that maintains a predetermined structure while containing a large amount of water, and is utilized in various medical applications due to biocompatibility and controlled drug release ability. In particular, a lidocaine-containing system using a hydrogel improves the sustained-release characteristic of the drug to sustain a long-term effect with a single administration. The lidocaine-containing system provides an advantage of reducing the inconvenience of repeated administration, maximizing the efficacy of the drug, and minimizing side effects.

The present disclosure relates to an injectable composition including a hydrogel containing lidocaine, and provides a technique for providing a sustained pain relief effect over a long period of time through stable sustained-release of lidocaine, and maximizing patient convenience and treatment efficiency.

### DISCLOSURE OF THE INVENTION

### Technical Goals

A technical aspect to be achieved by the present disclosure is to provide an injectable composition including a compound to form nanoparticles; a cross-linked hyaluronic acid hydrogel; and an anesthetic agent.

However, goals to be achieved by the present disclosure are not limited to those described above, and other goals not mentioned above can be clearly understood by one of ordinary skill in the art from the following description.

### Technical Solutions

According to an aspect, there is provided an injectable composition including a compound in which a compound represented by the following Chemical Formula 1 is conjugated with cyclodextrin-grafted polylysine (CDPL); a cross-linked hyaluronic acid hydrogel; and an anesthetic agent.

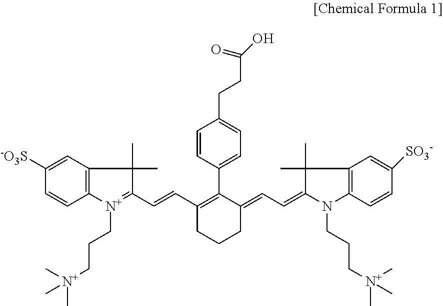

According to an embodiment, the anesthetic agent may be at least one selected from the group consisting of ambucaine, amolanone, amylocaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethysoquin, dimethocaine, diperodon, dycyclonine, ecgonidine, ecgonine, ethyl chloride, etidocaine, beta-eucaine, euprocin, fenalcomine, formocaine, hexylcaine, hydroxytetracaine, isobutyl paminobenzoate, leucinocaine mesylate, levoxadrol, lidocaine, mepivacaine, meprylcaine, metabutoxycaine, methyl chloride, myrtecaine, naepaine, octacaine, orthocaine, oxethazaine, parethoxycaine, phenacaine, phenol, piperocaine, piridocaine, polidocanol, pramoxine, prilocaine, procaine, propanocaine, proparacaine, propipocaine, propoxycaine, pseudococaine, pyrrocaine, ropivacaine, salicyl alcohol, tetracaine, tolycaine, trimecaine, zolamine, and salts thereof.

According to an embodiment, the anesthetic agent may be lidocaine.

According to an embodiment, the composition may be liquid at room temperature and gelated within a body temperature range.

According to an embodiment, the composition may sustained-release the anesthetic agent.

According to an embodiment, the cross-linked hyaluronic acid hydrogel may be a hydrogel cross-linked with hyaluronic acid of 50 to 150 kDa at a cross-linking degree of 20%.

According to another aspect, there is provided a method of preparing an injectable composition including the steps:
1) conjugating a compound represented by the following Chemical Formula 1 with CDPL;
2) succinylating and nanoparticleizing the compound conjugated in step 1);
3) preparing an anesthetic agent-nanoparticle solution by containing an anesthetic agent into nanoparticles prepared in step 2); and
4) mixing the anesthetic agent-nanoparticle solution into a cross-linked hyaluronic acid hydrogel.

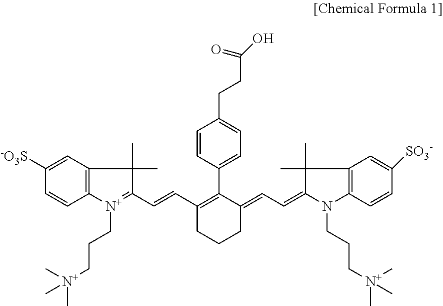

According to an embodiment, the anesthetic agent may be at least one selected from the group consisting of ambucaine, amolanone, amylocaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethysoquin, dimethocaine, diperodon, dycyclonine, ecgonidine, ecgonine, ethyl chloride, etidocaine, beta-eucaine, euprocin, fenalcomine, formocaine, hexylcaine, hydroxytetracaine, isobutyl paminobenzoate, leucinocaine mesylate, levoxadrol, lidocaine, mepivacaine, meprylcaine, metabutoxycaine, methyl chloride, myrtecaine, naepaine, octacaine, orthocaine, oxethazaine, parethoxycaine, phenacaine, phenol, piperocaine, piridocaine, polidocanol, pramoxine, prilocaine, procaine, propanocaine, proparacaine, propipocaine, propoxycaine, pseudococaine, pyrrocaine, ropivacaine, salicyl alcohol, tetracaine, tolycaine, trimecaine, zolamine, and salts thereof.

According to an embodiment, the anesthetic agent may be lidocaine.

According to an embodiment, the method may further include grinding the cross-linked hyaluronic acid hydrogel into a size suitable for injection, in step 4).

According to another aspect, there is provided a prefilled syringe filled with any one of the injectable compositions.

### Effects of the Invention

According to the present disclosure, the injectable composition is injected in a liquid formulation and then gelated in the body to prevent rapid decomposition, sustained-release the anesthetic agent for a long period of time, and locally remain in the body for a long period of time.

The effects of the present disclosure are not limited to the aforementioned effects, and other objects, which are not mentioned above, will be clearly appreciated by a person having ordinary skill in the art from the following description.

### BRIEF DESCRIPTION OF THE DRAWINGS

FIG. 1 shows rheological properties of a produced hydrogel formulation according to a temperature.

FIG. 2 shows results of confirming the degree of absorption of lidocaine as a local anesthetic agent in the body in a rat model.

FIG. 3 shows evaluation of in vivo safety of a hydrogel formulation and a lidocaine-containing hydrogel.

FIG. 4A shows the pain control effect of the lidocaine-containing hydrogel formulation.

FIG. 4B shows a comparison of the effects of the lidocaine-containing hydrogel formulation and the ropivacaine-containing hydrogel formulation.

### DETAILED DESCRIPTION FOR CARRYING OUT THE INVENTION

In order to solve the above problem, the present inventors provide an injectable composition including a compound in which a compound represented by the following Chemical Formula 1 is conjugated with cyclodextrin-grafted polylysine (CDPL); a cross-linked hyaluronic acid hydrogel; and an anesthetic agent.

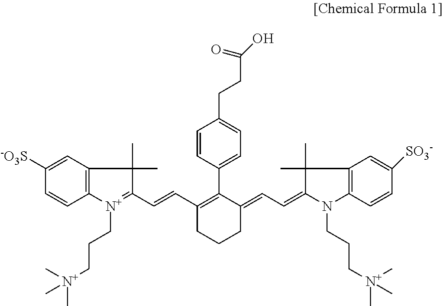

In the present disclosure, the compound in which the compound represented by Chemical Formula 1 is conjugated with CDPL forms nanoparticles to contain a drug lidocaine, and may act as a drug delivery system for lidocaine after released from the hydrogel.

In the case of using the compound in which the compound represented by Chemical Formula 1 is conjugated with CDPL, it was confirmed that the compound was suitable for long-term sustained release of the drug in the body by affecting drug release and rheological properties.

In addition, the composition of the present disclosure may be maintained in an injectable liquid formulation at room temperature and then gelated in a body temperature range after injected into the body. The body temperature range means 34 to 38° C. By gelling as described above, it is possible to prevent rapid decomposition and sustained-release the drug over a long period of time.

In the present disclosure, the anesthetic agent is not limited to any commonly used drug, but examples thereof include the following agents: ambucaine, amolanone, amylocaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethysoquin, dimethocaine, diperodon, dycyclonine, ecgonidine, ecgonine, ethyl chloride, etidocaine, beta-eucaine, euprocin, fenalcomine, formocaine, hexylcaine, hydroxytetracaine, isobutyl paminobenzoate, leucinocaine mesylate, levoxadrol, lidocaine, mepivacaine, meprylcaine, metabutoxycaine, methyl chloride, myrtecaine, naepaine, octacaine, orthocaine, oxethazaine, parethoxycaine, phenacaine, phenol, piperocaine, piridocaine, polidocanol, pramoxine, prilocaine, procaine, propanocaine, proparacaine, propipocaine, propoxycaine, pseudococaine, pyrrocaine, ropivacaine, salicyl alcohol, tetracaine, tolycaine, trimecaine, zolamine, and salts thereof. In addition, the anesthetic agent may be desirably lidocaine.

According to an embodiment, the composition may sustained-release the anesthetic agent.

According to an embodiment, the cross-linked hyaluronic acid hydrogel may be a hydrogel cross-linked with hyaluronic acid of 50 to 150 kDa at a cross-linking degree of 20%.

According to another embodiment of the present disclosure, there is provided a method of preparing an injectable composition including the following steps:
1) conjugating a compound of the following Chemical Formula 1 with CDPL;
2) succinylating and nanoparticleizing the conjugated compound of step 1);
3) preparing an anesthetic agent-nanoparticle solution by containing an anesthetic agent into nanoparticles prepared in step 2); and
4) mixing the anesthetic agent-nanoparticle solution into a cross-linked hyaluronic acid hydrogel.

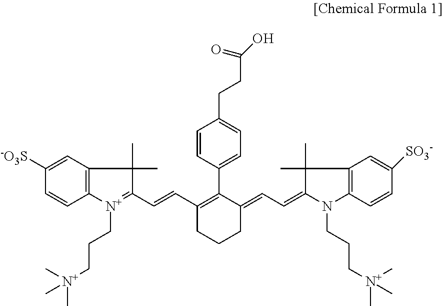

According to an embodiment, the method may further include grinding or homogenizing the cross-linked hyaluronic acid hydrogel into a size suitable for injection, in step 4).

According to yet another embodiment of the present disclosure, there is provided a prefilled syringe filled with any one of the injectable compositions.

The terms used in the embodiments are used for the purpose of description only, and should not be construed to be limited. A singular expression includes a plural expression unless otherwise defined differently in a context. In the present disclosure, it should be understood that term “including” or “having” indicates that a feature, a number, a step, an operation, a component, a part or the combination thereof described in the specification is present, but does not exclude a possibility of presence or addition of one or more other features, numbers, steps, operations, components, parts or combinations thereof, in advance.

Unless otherwise contrarily defined, all terms used herein including technological or scientific terms have the same meanings as those generally understood by a person with ordinary skill in the art to which embodiments pertain. Terms which are defined in a generally used dictionary should be interpreted to have the same meaning as the meaning in the context of the related art, and are not interpreted as ideal or excessively formal meanings unless otherwise defined in the present disclosure.

The present disclosure may have various modifications and various embodiments, and specific embodiments will be hereinafter illustrated in the drawings and described in detail in the detailed description. However, the present disclosure is not limited to specific embodiments, and it should be understood that the present disclosure covers all the modifications, equivalents and replacements within the idea and technical scope of the present disclosure. In describing the present disclosure, when it is determined that a detailed description of related known arts may obscure the gist of the present disclosure, the detailed description will be omitted.

### Example 1. Experimental Method

### Example 1-1. Preparation of Monoaldehyde β-Cyclodextrin (β-CD)

To prepare monoaldehyde β-CD (Ald-CD), 30 g of β-CD (26.4 mmol) was dissolved in 300 mL of anhydrous DMSO, and a round-bottom flask equipped with a stirring rod was used. Then, 13.5 g of Dess-Martin iodine (31.8 mmol) was added to the solution, completely dissolved, and then stirred at room temperature for 2 hours. Thereafter, the solution was precipitated in 3 L of EA/acetone (20% v/v) and stirred at room temperature overnight. The precipitate was recovered by vacuum filtration and then re-dissolved in a minimum amount of distilled water. The solution was sonicated for 20 minutes and then vacuum-filtered again. The filtered solution was frozen at −80° C. and then lyophilized. After lyophilizing, a white solid was obtained as a final product.

### Example 1-2. Conjugation of Ald-CD to EPL

20 g of Ald-CD (18 mmol) was dissolved in 700 mL of PBS (pH 8.0) to prepare a solution. Then, 7 g (1.8 mmol) of EPL was added, and the mixture was stirred for 24 hours. Thereafter, 7 g of sodium borate (180 mmol) was added to the reaction mixture to reduce a Schiff base to a secondary amine, and then further stirred for 24 hours at room temperature. Dialysis was performed by dynamic dialysis with deionized water for 72 hours using a 6-8 kDa molecular weight cutoff membrane. Finally, the dialyzed solution was frozen at −80° C. and then lyophilized.

### Example 1-3. Conjugation of NIR Phosphor to CDPL

First, ZW800 was prepared as follows.

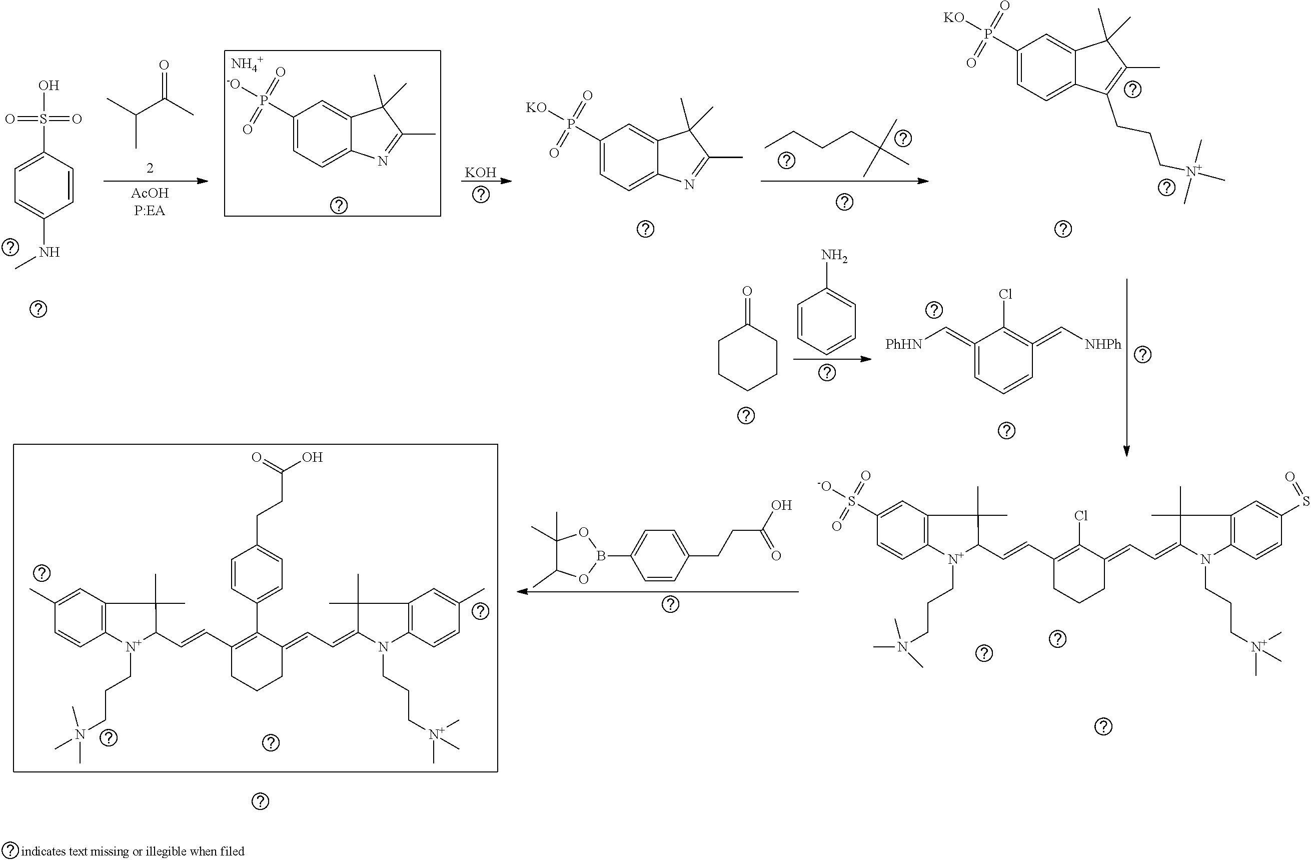

### 2,3,3-trimethyl-3H-indole-5-sulfonic Acid: Preparation of 4-hydrazinobenzenesulfonic Acid (20 g, 97.5 mmol)

Sodium acetate (16 g, 195 mmol) and 3-methyl-2-butanone (14.9 mL, 139 mmol) were mixed with glacial acetic acid (97 mL) and heated to 110° C. in a sealed tube under a nitrogen atmosphere. A produced crude product was filtered with a filter, washed with methyl tert-butyl ether (MTBE), and precipitated to obtain a brown solid (19.4 g, 83%).

### Preparation of 2,3,3-trimethyl-1-[3-(trimethylammonium) propyl]-3H-indolium-5-sulfonic acid

Dibromide: 2,3,3-trimethyl-3H-indole-5-sulfonic acid (7.17 g, 36.4 mmol) and (3-bromopropyl)trimethylammonium bromide (10.5 g, 40 mmol) were mixed in toluene (60 mL) and heated at 130° C. for 72 hours under a nitrogen atmosphere. After the mixture was cooled to room temperature, a solvent was decanted. The crude product was crystallized from methanol and MTBE to obtain pink crystals (5.11 g, 81%) in the next step without further purification.

### Preparation of 2,3,3-trimethyl-1-[3-(trimethylammonium) propyl]-3H-indolium bromide

2,3,3-trimethyl-3H-indole (1.59 g, 10 mmol) and (3-bromopropyl)trimethylammonium bromide (2.87 g, 11 mmol) were mixed in toluene (50 mL) and refluxed in a sealed tube under a nitrogen atmosphere for 72 hours. The reaction mixture was cooled to room temperature and concentrated under reduced pressure to obtain a red residue. The crude product was crystallized in acetone/methanol (5:1) to obtain a pink solid (2.52 g, 61%).

### Preparation of 2-((E)-2-((E)-2-chloro-3-((E)-2-(3,3-dimethyl-5-sulfonato-1-(3-(trimethylammonium) propyl) indolin-2-ylidene)ethylidene)cyclohex-1-en-1-yl) vinyl)-3,3-dimethyl-1-(3-(trimethylammonium)propyl)-3H-indolium-5-sulfonate disodium bromide

A bromide salt (1.26 g, 2.41 mmol), a Vilsmeier-Haack reagent (0.433 g, 1.5 mmol), and anhydrous sodium acetate (0.37 g, 4.5 mmol) were mixed in absolute ethanol (50 mL), and refluxed under a nitrogen atmosphere for 6 hours. The reaction mixture was cooled to room temperature and concentrated under reduced pressure to obtain a brown residue. The crude product was washed with dichloromethane to obtain a brown-green solid, and the brown-green solid was suspended in methanol (10 mL), filtered, and dried under vacuum to obtain a golden-green solid (1.2 g, 1.1 mmol, 73%).

### Preparation of 3-((E)-2-(3,3-dimethyl-1-(3-(trimethylammonium) propyl) indolin-2-ylidene)ethylidene)cyclohex-1-en-1-yl) vinyl)-3,3-dimethyl-1-(3-(trimethylammonium) propyl)-3H-indolium bromide

A bromide salt (840 mg, 2 mmol), a Vilsmeier-Haack reagent (359 mg, 1 mmol), and anhydrous sodium acetate (492 mg, 6 mmol) were mixed in absolute ethanol (50 mL), and refluxed at 100° C. for 5 hours. The mixture was cooled to room temperature and concentrated under reduced pressure to obtain a brown residue. The reaction mixture was dried, and then the brown residue was washed with dichloromethane and MTBE (1:1) to obtain a dye (260 mg, 29%).

Synthesis of C—C linked ZW800 analogue: 3-((E)-2-(3,3-dimethyl-1-(3-(trimethylammonium) propyl) indolin-2-ylidene)ethylidene)cyclohex-1-en-1-yl) vinyl)-3,3-dimethyl-1-(3-(trimethylammonium) propyl)-3H-indolium bromide (1.0 mmol) and 3-(4-boronophenyl) propionic acid (1.8 mmol) were added to H₂O and refluxed for 72 hours in the presence of Pd(PPh3)4 (0.065 mmol). The reaction was monitored by visible/near-infrared spectroscopy using methanol-diluted samples and performed until the absorption of a starting material disappeared. The mixture was cooled to room temperature and H₂O was removed under reduced pressure. The solid was separated by precipitation with methanol/acetone, and the precipitate was further washed with acetone. A final fluorescent material of analytical purity was obtained using open-reverse phase column chromatography (eluted with acetonitrile/water).

After the synthesis of ZW800-1C as described above, a solution of ZW800-1C-NHS ester was prepared by dissolving 22.9 mg (22.38 μmol) of an NIR phosphor in 1 mL of DMSO. A CDPL solution was prepared by dissolving 250 mg (18.65 μmol, MW 13,403) of CDPL in 25 mL of PBS (pH 9.0) in a round-bottom flask equipped with a stirring rod. The ZW800-1C-NHS ester solution was slowly added dropwise into the CDPL solution with vigorous stirring. The reaction mixture was stirred for 3 hours, and 0.6 M sodium hydroxide was added as needed while maintaining pH at 9.0. After 3 hours, the product was precipitated in 250 mL of EA/acetone (20% v/v) and centrifuged at 3000 rpm for 15 minutes. After the supernatant was discarded, a process of dissolving ZW800-CDPL in DIW and precipitating again in EA/acetone was repeated twice more. The product was dried under vacuum to obtain a green solid.

### Example 1-4. Succinylation of ZW800-CDPL

To prepare amphoteric nanoparticles (NPs), 2 g (143 μmol) of ZW800-CDPL was dissolved in PBS (pH 9.0; 10 mg/mL). Then, 300 mg of a succinic anhydride (3 mmol) solution dissolved in DMSO (250 mg/mL) was added. The mixture was stirred at room temperature for 1 hour, and added with 0.6 M sodium hydroxide as needed to maintain the pH at 9.0. After confirming partial succinylation by a ninhydrin test, the product was dialyzed with DIW for 72 hours. After dialysis, the solution was frozen at −80° C. and then lyophilized.

### Example 1-5. Preparation of Lidocaine Nanoparticles (Lid-NPs)

To prepare Lid-NPs, 90 mg of lidocaine was dissolved in DIW. Thereafter, 500 mg of NP powder was dissolved in DIW, and 90 mg of lidocaine was dissolved in an NP solution. The mixture was shaken at room temperature for 2 hours. Thereafter, the solution was centrifuged at 14,000 rcf for 10 minutes to precipitate impurities, and filtered through a spin column (MWCO 10K) to obtain a product complex in the supernatant. The molar ratio of lidocaine to NPs was measured using a UV spectrophotometer.

### Example 1-6. Preparation of Cross-Linked Hyaluronic Acid (HA)

100 mg of HA was dissolved in 600 μL of a 0.3 M NaOH solution. 20 μL of 1,4-butanediol diglycidyl ether (BDDE) was added to the HA solution. The mixture was shaken for 1 minute and reacted at 40° C. for 2 hours. Thereafter, the mixture was neutralized to pH 7.0 with 0.1 M HCl. The neutralized reaction mixture was dialyzed with deionized water (DW) using a 6-8 kDa molecular weight cutoff membrane to remove the remaining BDDE. After dialysis, the HA hydrogel was broken down into injectable particle sizes using a syringe with an 18-23 G needle. The injectable HA hydrogel particles (xHA) were lyophilized.

### Example 1-7. Preparation of Injectable Hydrogel Loaded with Lid-NPs

Lid-NPs were dissolved in DW at a concentration of 1 g mL⁻¹. 70 mg of xHA was added to 300 μL of a Lid-NP solution to prepare Lid-NP-loaded xHA. Lid-NP-loaded xHA was lyophilized. The lyophilized Lid-NP-loaded xHA was added to 10 wt % of a Pluronic F127 solution on ice before use. The composition of the injectable hydrogel loaded with Lid-NP was as shown in Table 1 below.

TABLE 1

| | Lid-NP hydrogel |
|---|---|
| Loaded drug | Lidocaine |
| Composition | 7% xHA |
| | 10% F127 |
| | 1-5% Lid-NP |

### Example 2. Confirmation of Change in Rheological Properties of Lidocaine-Containing Hydrogel

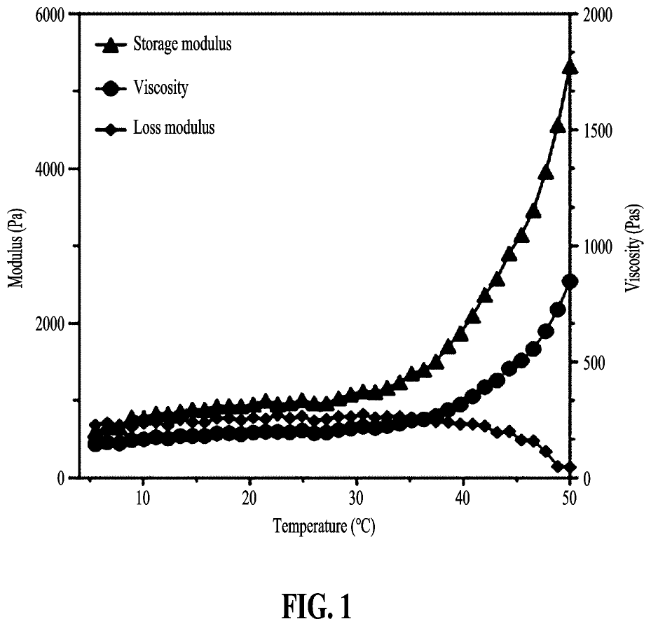

The rheological properties of a produced hydrogel formulation according to a temperature were confirmed and shown in FIG. 1. The rheological changes to be gelated in an environment of 34° C. to 36° C. were confirmed. As a result, it was confirmed that the hydrogel produced through the study could change from a liquid state to a gel form and maintain the change through a contact with an environment similar to a body temperature.

### Example 3. Pharmacodynamic Evaluation of Lidocaine-Containing Hydrogel

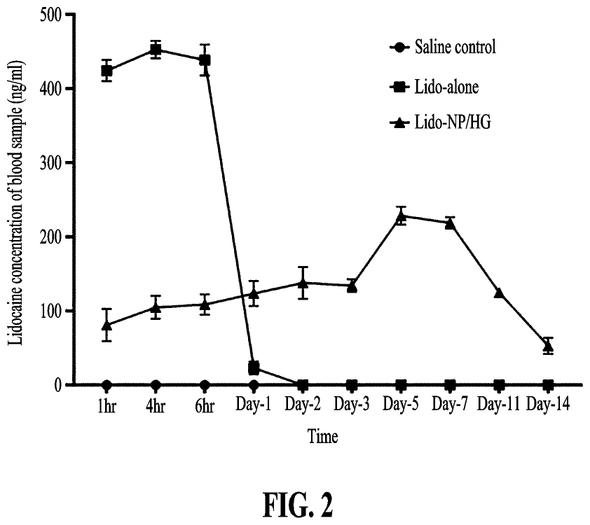

The degree of absorption of a lidocaine local anesthetic agent in the body was confirmed through the serums in a rat model injected with lidocaine alone and a rat model injected with a lidocaine-containing hydrogel, which was shown in FIG. 2. As a result, it was confirmed that in the serum of animals treated with lidocaine alone, lidocaine was measured for 1 hr to 6 hr, and it was confirmed that in animals injected with a lidocaine-containing hydrogel, the concentration of lidocaine in the serum was measured at a low concentration for a long period of time (1 hr to 14 days) without excessive measurement within a short period of time after injection. It is shown that the lidocaine-containing hydrogel invented in this study sustained-releases lidocaine slowly in vivo.

### Example 4. Evaluation of In Vivo Safety of Hydrogel Formulation and Lidocaine-Containing Hydrogel

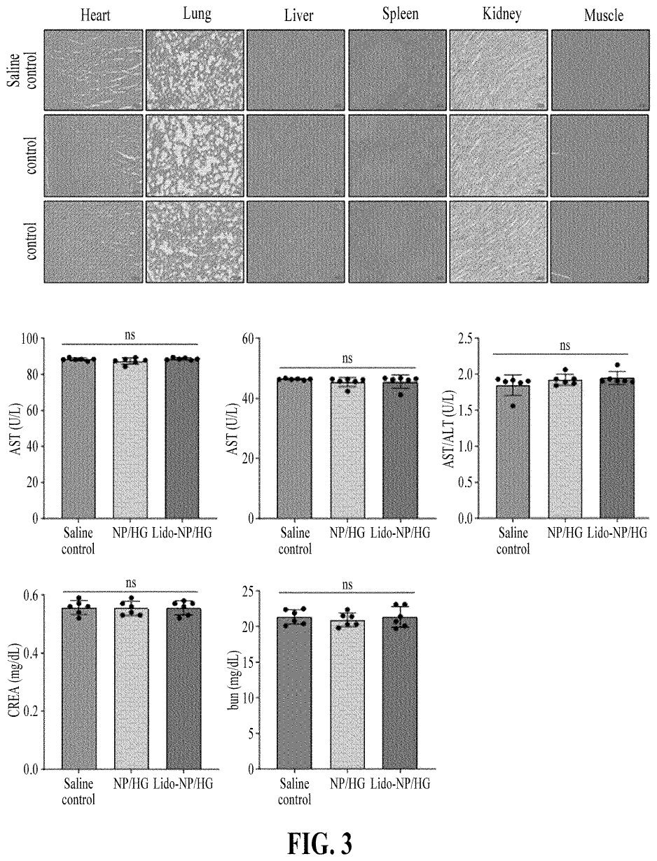

The effects of the injection of a hydrogel without containing lidocaine and a hydrogel containing lidocaine on major organs in vivo were confirmed histologically, and simultaneously, the liver damage and myocardial damage scales that may be confirmed at the blood level were confirmed through blood pathology, which were shown in FIG. 3.

As a result, it was confirmed that the major organ pathological analysis of the rat animal models injected with the hydrogel and the lidocaine-containing hydrogel did not affect the tissue damage and pathological safety compared to a control group. In addition, even in the blood analysis collected after hydrogel injection, when indicators of liver damage and myocardial damage were confirmed, it was confirmed that no significant changes were observed compared to the control group.

### Example 5. Evaluation of Pain Control Effect of Lidocaine-Containing Hydrogel

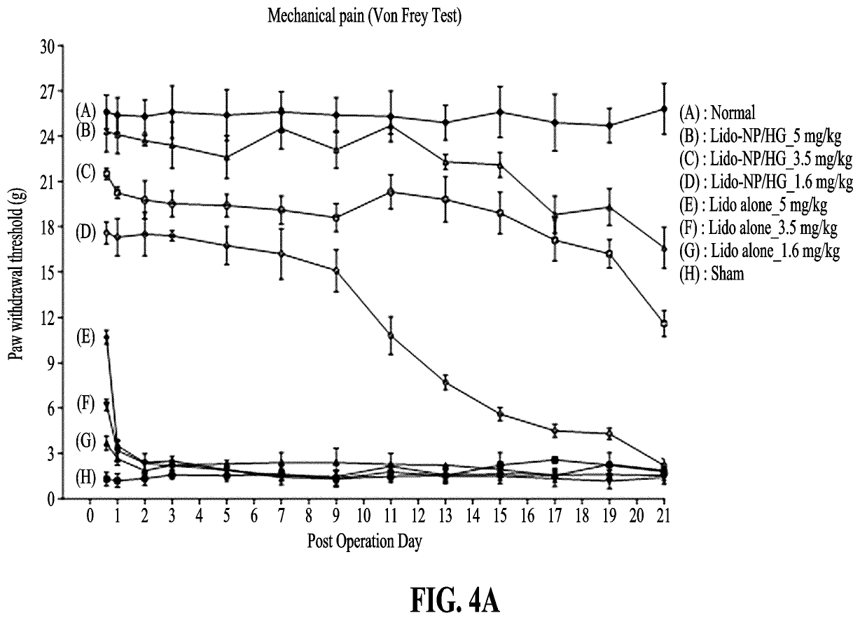

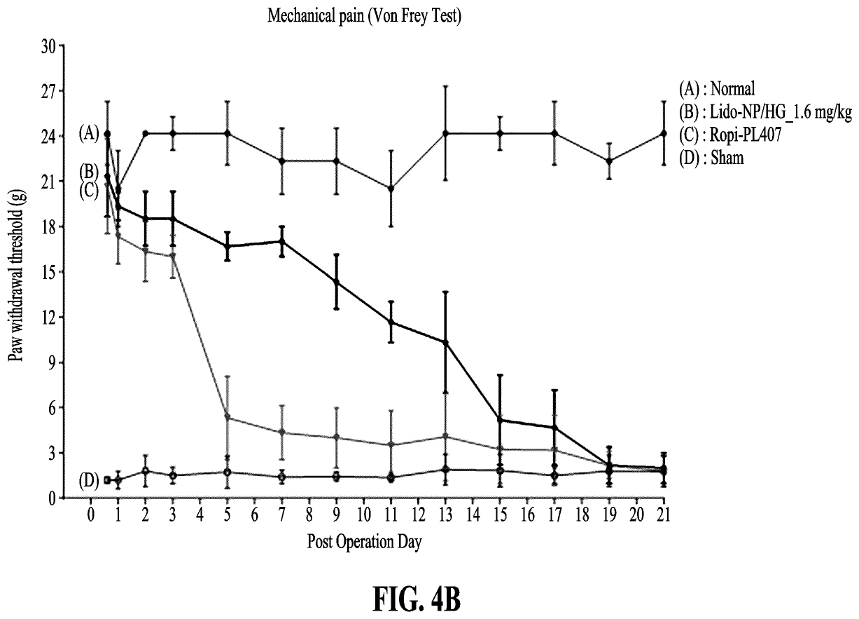

The results of confirming a pain control effect of a lidocaine-containing hydrogel formulation were shown in FIG. 4A. An animal model was confirmed using a rat chronic constriction injury (CCI) model, which was generally used for a pain sensitivity experiment. As shown in a graph of FIG. 4A, a pain model was fabricated through rat nerve ligation, lidocaine alone was injected around the nerve tissue at a concentration of 1.6 mg/kg to 5 mg/kg, and the hydrogel containing lidocaine at the same concentration was injected using the same method. As a result, it was confirmed that the pain control effect was significantly increased in a group injected with the hydrogel containing lidocaine compared to an animal model injected with lidocaine alone. In addition, as a result of comparing the pain control effect with a Ropivacaine-hydrogel formulation that has already been developed and used clinically through an experiment of FIG. 4B, it was confirmed that the injection of the lidocaine-containing hydrogel invented in the present disclosure may stably maintain the pain control effect in the pain model for up to 14 days, compared to the Ropivacaine-hydrogel that may maintain the pain control effect for up to 3 days.

As described above, although the examples have been described by the restricted drawings, various modifications and variations may be applied on the basis of the embodiments by those skilled in the art. For example, even if the described techniques are performed in a different order from the described method, and/or components such as a system, a structure, a device, a circuit, and the like described above are coupled or combined in a different form from the described method, or replaced or substituted by other components or equivalents, an appropriate result may be achieved.

Therefore, other implementations, other preparation examples, and equivalents to the appended claims fall within the scope of the claims to be described below.

1. An injectable composition comprising:
a compound in which a compound represented by the following Chemical Formula 1 is conjugated with cyclodextrin-grafted polylysine (CDPL);
a cross-linked hyaluronic acid hydrogel; and
an anesthetic agent.

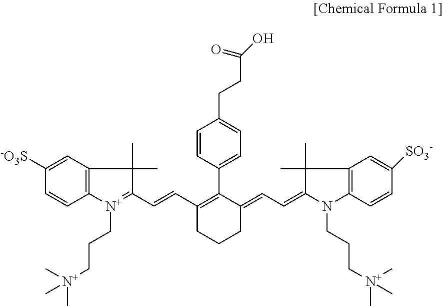

2. The injectable composition of claim 1, wherein the anesthetic agent is at least one selected from the group consisting of ambucaine, amolanone, amylocaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethysoquin, dimethocaine, diperodon, dycyclonine, ecgonidine, ecgonine, ethyl chloride, etidocaine, beta-eucaine, euprocin, fenalcomine, formocaine, hexylcaine, hydroxytetracaine, isobutyl paminobenzoate, leucinocaine mesylate, levoxadrol, lidocaine, mepivacaine, meprylcaine, metabutoxycaine, methyl chloride, myrtecaine, naepaine, octacaine, orthocaine, oxethazaine, parethoxycaine, phenacaine, phenol, piperocaine, piridocaine, polidocanol, pramoxine, prilocaine, procaine, propanocaine, proparacaine, propipocaine, propoxycaine, pseudococaine, pyrrocaine, ropivacaine, salicyl alcohol, tetracaine, tolycaine, trimecaine, zolamine, and salts thereof.

3. The injectable composition of claim 2, wherein the anesthetic agent is lidocaine.

4. The injectable composition of claim 1, wherein the composition is liquid at room temperature and gelated within a body temperature range.

5. The injectable composition of claim 1, wherein the composition sustained-releases the anesthetic agent.

6. The injectable composition of claim 1, wherein the cross-linked hyaluronic acid hydrogel is a hydrogel cross-linked with hyaluronic acid of 50 to 150 kDa at a cross-linking degree of 20%.

7. A method of preparing an injectable composition, the method comprising the steps:
1) conjugating a compound represented by the following Chemical Formula 1 with cyclodextrin-grafted polylysine (CDPL);
2) succinylating and nanoparticleizing the compound conjugated in step 1);
3) preparing an anesthetic agent-nanoparticle solution by containing an anesthetic agent into nanoparticles prepared in step 2); and
4) mixing the anesthetic agent-nanoparticle solution into a cross-linked hyaluronic acid hydrogel.

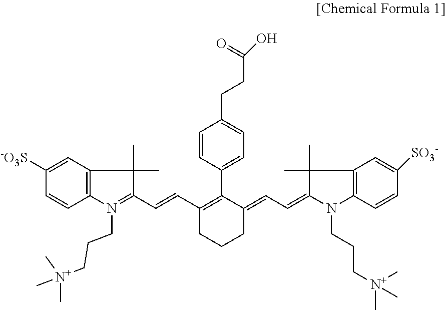

8. The method of claim 7, wherein the anesthetic agent is at least one selected from the group consisting of ambucaine, amolanone, amylocaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethysoquin, dimethocaine, diperodon, dycyclonine, ecgonidine, ecgonine, ethyl chloride, etidocaine, beta-eucaine, euprocin, fenalcomine, formocaine, hexylcaine, hydroxytetracaine, isobutyl paminobenzoate, leucinocaine mesylate, levoxadrol, lidocaine, mepivacaine, meprylcaine, metabutoxycaine, methyl chloride, myrtecaine, naepaine, octacaine, orthocaine, oxethazaine, parethoxycaine, phenacaine, phenol, piperocaine, piridocaine, polidocanol, pramoxine, prilocaine, procaine, propanocaine, proparacaine, propipocaine, propoxycaine, pseudococaine, pyrrocaine, ropivacaine, salicyl alcohol, tetracaine, tolycaine, trimecaine, zolamine, and salts thereof.

9. The method of claim 8, wherein the anesthetic agent is lidocaine.

10. The method of claim 7, further comprising:
grinding the cross-linked hyaluronic acid hydrogel into a size suitable for injection, in step 4).

11. A prefilled syringe filled with the composition of claim 1.
